(19) United States
(12) Patent Application Publication
(10) Pub. No.: US 2026/0016456 A1
(43) Pub. Date: Jan. 15, 2026

(54) WATER TOXICITY DETECTION SYSTEM AND METHOD

(71) Applicant: John Carson, Morrison, CO (US)

(72) Inventor: John Carson, Morrison, CO (US)

(21) Appl. No.: 19/264,063

(22) Filed: Jul. 9, 2025

Related U.S. Application Data
(60) Provisional application No. 63/669,031, filed on Jul. 9, 2024.

(51) Int. Cl. G01N 33/18

(52) U.S. Cl. CPC G01N 33/186

(57) ABSTRACT

A water toxicity detection system and method employ bivalve organisms as biological indicators to monitor aquatic environments in real-time. The system includes sensors configured to measure gape behavior of multiple bivalve organisms, generating corresponding gape measurements that are processed by a computing system. The processor normalizes gape measurements and calculates exponentially weighted moving average (EWMA) and exponentially weighted moving variance (EWMV) values to assess short-term behavioral patterns. A detection module uses EWMA and EWMV as state-space variables to identify deviations indicative of exposure to toxic substances, specifically detecting gape closing (GC) events characterized by increased activity followed by shell closure. The system generates system-level alarms when a predetermined fraction of individual bivalves simultaneously exhibits abnormal behavior patterns consistent with toxicity exposure. The technology enables early detection of waterborne contaminants including heavy metals, organic compounds, industrial chemicals, and algal toxins.

### CROSS-REFERENCE TO RELATED APPLICATIONS

This application claims the benefit of U.S. Provisional Application No. 63/669,031, filed on Jul. 9, 2024. The entire disclosure of the above application is incorporated herein by reference.

### FIELD

The present technology relates to environmental monitoring systems and, more particularly, to biological early warning systems for the detection of aquatic toxicity using a bivalve organism.

### INTRODUCTION

This section provides background information related to the present disclosure which is not necessarily prior art.

Aquatic environments may frequently experience chemical pollution, which may come from a point source (e.g., governmental or industrial wastewater) or a non-point source (e.g., airborne deposition, runoff from urban or agricultural regions, or algae bloom). It may be essential to protect drinking water sources from such contamination, the occurrence of which may often be sporadic and unpredictable. Situations of accidental or deliberate contamination must be detected rapidly and a speedy response coordinated, owing to the potential for serious impacts for human health. Consequently, early warning monitoring may be used to rapidly identify an adverse water quality change.

Chemical sensors and periodic sampling for chemical analysis may be used in monitoring of aquatic environments. However, chemical sensors may be expensive to maintain, specific to only certain pollutants, and reveal only local, short-term water quality changes. Moreover, many chemical spill events may occur sporadically and contain unpredicted pollutants, and as such, may go undetected. Chemical monitoring of aquatic environments may be further hindered by the wide diversity of chemical classes that may be present. Additionally, chemical analytical techniques may not fully capture the toxic effects of chemical mixtures which may be additive, synergistic, or antagonistic. Periodic sampling and analysis using laboratory methods may be quite expensive. The cost to analyze one water sample for the list of 126 U.S. Environmental Protection Agency (EPA) priority pollutants may be prohibitively expensive.

The complexity, in aquatic environments, of detecting and quantifying a wide array of toxic pollutants continuously, in real-time, including both emerging pollutants and transformation products, combined with the inability to measure toxicity directly, has led to the development and employment of biomonitors. Biological early warning systems (BEWSs) monitor the responses of organisms, based on behavior or physiology, allowing continuous sensing of a wide range of pollution. In addition to the test organisms, BEWSs have two further components: an automated detection system, and an alarm system. Hence, altered responses, resulting from pollution, may be rapidly detected and the alarm raised, allowing appropriate action to be taken. The same issues and solutions may apply to the somewhat lesser problems of protection of recreational waters, protection of aquaculture, and protection of sensitive ecosystems.

BEWs for real-time toxicity detection may include a variety of classes of organisms using a variety of different detection principles. These may include genetically engineered bacteria (e.g., bioluminescence), algae (e.g., reduced oxygen production and production of hydrogen peroxide and other reactive oxygen species), small crustaceans (e.g., amphipods and daphnids, using behavior and mortality), bivalves (e.g., clams and mussels, using gape, heart rate, and mortality), and minnows (e.g., behavior and mortality). These systems may have varying effectiveness in terms of range of toxins detected, sensitivity, response time, and false alarm rates, but BEWs using organisms other than bivalves require a lot of maintenance. Bioluminescence-based detection systems require specific knowledge of biochemical pathways of toxicity and the corresponding engineering of specific bacterial strains for each chemical toxin monitored. However, such systems may be complicated and may require further computational and analytical analysis to evaluate the behavioral data of the organisms.

Accordingly, there is a need for a system and method for detecting toxicity in surface waters in near real-time using an algorithm applied to measurements of bivalve gape.

### SUMMARY

In concordance with the instant disclosure, a system and method for detecting toxicity in surface waters in near real-time using an algorithm applied to measurements of bivalve gape, has surprisingly been discovered. The present technology includes articles of manufacture, systems, and processes that relate to the real-time detection and monitoring of aquatic toxicity through the use of bivalve organisms as bioindicators, sensors for measuring their gape behavior, and advanced computational methods for data analysis.

In certain embodiments, a system for detecting toxicity in an aquatic environment can comprise an enclosure that can be configured to contain water from the aquatic environment, with a plurality of bivalve organisms that can be positioned within the enclosure. The system can include a plurality of sensors that can be positioned within the enclosure, where each sensor can be configured to measure a gape of an individual bivalve organism and can generate corresponding gape measurements for that individual bivalve organism. A processor can be configured to normalize the gape measurements from each individual bivalve organism to a predefined interval and can calculate, for each individual bivalve organism, an exponentially weighted moving average (EWMA) and an exponentially weighted moving variance (EWMV) of the normalized gape. The system can further include a detection module that can be configured to use the EWMA and EWMV as state-space variables to monitor behavior of each individual bivalve organism and can identify a deviation in behavior of each individual bivalve organism that can be indicative of exposure to a potentially toxic substance. An alarm system can be configured to generate a system-level alarm when a predetermined fraction of the individual bivalve organisms exhibits the identified deviation, enabling early detection and response to environmental contamination events.

In certain embodiments, a method of detecting toxicity in an aquatic environment can comprise positioning a plurality of bivalve organisms within an enclosure that can be configured to contain water from the aquatic environment and can include obtaining a gape measurement from each individual bivalve organism using a plurality of sensors that can be positioned within the enclosure. The method can include normalizing the gape measurement from each individual bivalve organism to a predefined interval and can include calculating, for each individual bivalve organism, an exponentially weighted moving average (EWMA) and an exponentially weighted moving variance (EWMV) of the normalized gape. The method can further comprise monitoring behavior of each individual bivalve organism using the EWMA and EWMV as state-space variables, can include identifying a deviation in behavior of individual bivalve organism that can be indicative of exposure to a potentially toxic substance, and can include generating a system-level alarm when a predetermined fraction of the individual bivalve organisms exhibit the identified deviation. This method can provide real-time monitoring capabilities that can detect environmental contamination through the natural biological responses of bivalve organisms to changes in water quality.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a block diagram showing certain aspects of a system for detecting toxicity in an aquatic environment, according to an embodiment of the present disclosure.

FIG. 2 is a flowchart illustrating a method of real-time monitoring of water toxicity using bivalve organisms, according to an embodiment of the present disclosure.

FIG. 3 is a correlation table for normalized gape measurements across multiple bivalve organisms, according to an embodiment of the present disclosure.

FIG. 4 is a graphical representation of an autocorrelation function for a normalized gape, according to an embodiment of the present disclosure.

FIG. 5 is a graphical representation of a partial autocorrelation function for a normalized gape, according to an embodiment of the present disclosure.

FIG. 6 is a table of estimated ARMA(1,1) coefficients for normalized gape measurements, according to an embodiment of the present disclosure.

FIG. 7 is a graphical representation of a bivalve gape closing event, according to an embodiment of the present disclosure.

FIG. 8 is a graphical representation of a bivalve gape closing event shown in an Exponentially Weighted Moving Average (EWMA) and an Exponentially Weighted Moving Variance (EWMV) state space plot, according to an embodiment of the present disclosure.

FIG. 9 is a table showing estimated effect of bivalve organism on GC event probability, showing individual organism differences in response probability, according to an embodiment of the present disclosure.

FIG. 10 is a table showing estimated effect of hour of day on GC event probability, according to an embodiment of the present disclosure.

FIG. 11 is a correlation table for MWW GC event indicator variables, according to an embodiment of the present disclosure.

FIG. 12 is a graphical representation of an autocorrelation function for a bivalve gape closing event, according to an embodiment of the present disclosure.

FIG. 13 is a graphical representation of an autocorrelation function for a bivalve gape closing event ratio, according to an embodiment of the present disclosure.

FIG. 14 is a graphical representation of a histogram of a bivalve gape closing event ratio, according to an embodiment of the present disclosure.

FIG. 15 is a graphical representation of an empirical cumulative distribution function for a bivalve gape closing event ratio, according to an embodiment of the present disclosure.

FIG. 16 is a table showing effect of hour of day on MWW GC event ratio, according to an embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for case of description to describe one clement or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology relates to a system and method for detecting toxicity in surface waters in near real-time using an algorithm applied to measurements of bivalve gape. As used herein, the term “gape” can refer to the degree of shell opening in a bivalve organism, representing the physical separation between the two shell halves at any given time. Under normal environmental conditions, a bivalve can maintain its shell halves to allow for feeding and respiration, as the filter-feeding organism can extract food particles and oxygen from the surrounding water. However, gape behavior can serve as a biological indicator for water quality monitoring because the bivalve can alter a shell opening pattern in response to an environmental stressor, including the presence of one or more toxic substances in the water. The bivalve can also be sacrificed periodically to monitor bioaccumulation of one or more pollutants and replaced with an acclimated bivalve.

When exposed to potentially harmful contaminants, the bivalve can typically exhibit characteristic behavioral changes in a gape pattern, including increased activity (e.g., rapid opening and closing movements, possibly attempting to flush gills) followed by partial or complete shell closure as an escape response. This natural defensive behavior can make gape measurement an effective real-time biomonitoring parameter, as changes in gape patterns can indicate the presence of waterborne toxins before more severe biological effects occur, enabling early detection and response to environmental contamination events. Individual level alarms can be generated for individual bivalves, based on crossing certain thresholds. When a sufficient fraction of the individual bivalves is in the alarm state, a system level alarm can be triggered. For example, a toxicity signal can be conveyed to responsible personnel, such as on-duty water treatment plant laboratory personnel, by audiovisual alarm, text message, and/or email. A toxicity signal can optionally trigger automated sampling.

The system can employ indigenous bivalve species that are specifically selected based on the local aquatic environment to be monitored. For freshwater applications, the system may utilize species from the family Unionidae, with Elliptio complanta being suitable, or species like Dreissena polymorpha (zebra mussel) and Corbicula manilensis (Asiatic clam) that are already present in a given local environment. For saltwater applications, the system can employ diverse bivalve species including Mytilus species (marine mussels), including Mytilus edulus (blue mussels), Crassostrea species (oysters), and species of the family Pectinidae (scallops). The use of an indigenous bivalve species avoids introducing non-native species into the environment and ensures that the selected organisms are already adapted to the local environmental conditions, making the organism more sensitive and responsive to changes in water quality that could indicate toxic contamination. Advantageously, the use of the indigenous species can enhance the effectiveness of the system because the organism is naturally suited to detect deviations from normal environmental conditions in the native habitat of the organism.

The system can monitor for a wide range of toxic substances and contaminants in both freshwater and marine environments. The system can detect heavy metals including cadmium (Cd) at concentrations as low as 50 μg/L, copper (Cu) at 5-10 μg/L, mercury (Hg) at 10 μg/L, lead (Pb) at concentrations above 100 μg/L, and zinc (Zn) at 10-500 μg/L depending on the environment. The system can also monitor for organic compounds such as chlorpyrifos at 50μg/L, pentachlorophenol at 10-50 μg/L, tributylin oxide at 6-10 μg/L, and hexachlorohexane at 6μg/L. Additionally, the system can detect industrial chemicals including chloroform at 43,000 μg/L, dichloromethane at 50,000 μg/L, dispersed crude oil at 250-6,000 μg/L, and various other organic solvents and compounds. The system can also respond to algal toxins, including microcystins at concentrations of 0.5-50 μg/L, saxitoxin equivalents at 0.1-500 μg/L, and okadaic acid at 50-150 ng/L, making the system effective for detecting harmful algal bloom events. It should be appreciated that the contaminants and concentrations denoted herein are non-limiting examples, and that there may be variations in the detection abilities of the particular bivalve selected.

A system for detecting toxicity in an aquatic environment can be provided. In certain embodiments, the system can include one or more bivalve organisms, one or more sensors, a processor or analysis module, a detection module, and an alarm system. The system can be configured to operatively communicate with a computing device. In certain embodiments, a sensor or multiple sensors can be configured to measure a gape or opening of the bivalve organism and generate a corresponding gape measurement. Each individual bivalve can be equipped with a dedicated sensor to enable monitoring of shell movement and gape behavior of multiple bivalves at an individual level. The sensors work in conjunction with magnets to detect the position of the bivalve shell halves based on electromagnetic field variations. The sensing apparatus can include a movable member that engages the shell of the mollusk and moves when the shell opens and closes, with a Hall effect sensor detecting the position based on the movement of this member, for example. An adhesive system can hold the sensor firmly in place but can allow the sensor to be placed and removed without harming the bivalve.

The sensor can generate a gape measurement that represents the degree of shell opening. The raw gape measurement can vary between 0 and a large positive integer, often exceeding 2500 units. The analog output from the sensor can be converted to digital form through an analog/digital converter that can interrogate the sensor at a frequency of 2 to 10 Hz, meaning once every half second to one tenth of a second. The digital data can be logged to a disk drive or transmitted to a remote location, with calculations preferably performed about once every minute.

The system can include a sensing enclosure for the bivalves that can allow uniform upflow of water being monitored across the bivalves with valving and a flow meter to adjust and control the flow of water to a predetermined velocity. The enclosure can be configured to ensure consistent water flow conditions across all monitored bivalves, enabling accurate comparative measurements of their gape behavior. The flow control system can allow operators to optimize water velocity for the specific bivalve species and environmental conditions being monitored. The enclosure can be integrated directly into existing water treatment facility piping systems or can be operated as a standalone monitoring unit.

The Hall effect sensor can employ an amplifier system, with an amplifier card that can accommodate multiple sensors, for examples, four sensors can be connected to a single amplifier card. For a system with 16 bivalves, the hardware can include a box with four amplifier cards installed in an environmentally sealed enclosure to protect against moisture. The amplification system can provide linear output over the required range of voltages at very low current levels and small voltages characteristic of Hall effect sensor operation. The system can also incorporate a reference sensor placed in the same enclosure of the bivalves and connected to the same signal amplifier and data acquisition hardware as the bivalves. The reference sensor can include a magnet and a Hall sensor separated by a non-conductive block, positioned in the same tank with the bivalves. The reference sensor can be used to adjust the raw gape signal from the bivalves to reduce extraneous electromagnetic noise and correct for stray voltages in the tank as well as voltage fluctuations in the amplifier. The reference system can help mitigate against electronic noise and possible impact of stray current in the water that could affect the magnetic field measurements.

The system can incorporate one or more auxiliary sensors configured to measure water quality parameters including sensors for temperature, dissolved oxygen, pH, specific conductance, turbidity, and UV/VIS absorbance bands for chlorophyll detection. These auxiliary sensor measurements can serve as additional inputs to enhance the accuracy of toxicity detection and can be used to guide chemical testing in response to a signal of toxicity. The auxiliary sensors can provide environmental context that helps distinguish between normal behavioral variations due to environmental changes and abnormal behavior indicative of toxic exposure, enabling facility personnel to make more informed decisions about appropriate response measures and chemical testing protocols. The system can continuously monitor these parameters alongside the bivalve gape measurements to provide a comprehensive assessment of water quality conditions.

Optional elements of the hardware system can include a preconditioning chamber for adjusting water temperature to an optimal range and for adding supplemental food if needed for monitoring water with an insufficient amount of phytoplankton for sustaining the bivalves. The system can also include one or more additional chemistry sensors, for instance a colorimeter or UV/VIS spectrometer, and an additional holding tank or chamber for conditioning bivalves held in reserve as replacements for those that die, are retired, or are sacrificed to monitor bioaccumulation of pollutants. These reserve bivalves can be exposed to the same stream of water as the bivalves in the sensing enclosure.

The system can identify abnormal behavior through detection of a gape closing (GC) event, which can exhibit a characteristic statistical pattern in the EWMA/EWMV state-space. A GC event can be characterized by an initial substantial increase in the EWMV accompanied by a decreasing EWMA, followed by both the EWMA decreasing to a small value and the EWMV approaching near zero. This behavioral sequence can create a distinctive trajectory path in the statistical state-space that can be automatically detected and can serve as an indicator of potential toxic exposure. The signature pattern can reflect the natural response of the bivalve to contamination: increased shell activity (e.g., captured by rising EWMV) as the organism attempts to flush its gills, followed by shell closure (e.g., reflected in low EWMA and EWMV values) as a protective response. The system can use predefined thresholds in the EWMA/EWMV state-space to automatically identify when individual bivalves exhibit this characteristic GC event pattern.

The multi-bivalve approach can be important because individual bivalves can have different sensitivities and may occasionally exhibit abnormal behavior for non-toxic reasons. Individual bivalves can have significantly different baseline signaling frequencies, with some being much more sensitive than others. By requiring multiple bivalves to simultaneously exhibit the abnormal GC event pattern, the system can distinguish between individual behavioral variations and actual environmental contamination events. The system can calculate a “GC event ratio” representing the ratio of bivalves in a GC event state to the number of active clams. The system can also account for the fact that sometimes bivalves can close and become quiescent or simply be open but fail to change gape for several days, and therefore can distinguish between active and inactive bivalves when calculating system-level alarms. When the ratio exceeds predetermined thresholds, the system can trigger a toxicity alert, ensuring that individual behavioral variations do not result in false system-level alarms.

This statistical approach can allow the system to provide early warning of water contamination while minimizing false alarms, as it can be highly unlikely that multiple bivalves would simultaneously exhibit the characteristic toxic response pattern unless there was actually something harmful in the water. The correlation analysis shows that while individual bivalves can exhibit different sensitivities, there can appear to be significant correlation between organisms which seems most likely due to similar responses to changing common environmental conditions.

The system can determine which organisms exhibit normal patterns of behavior versus inactivity at maximum gape in normal environmental conditions, which may indicate illness or death. The active/inactive status of the bivalves can be assessed each day at midnight using specific criteria to distinguish between responsive and non-responsive organisms. If the range of a bivalve's raw gape measurements over the course of a day does not change by more than 10 units (about 0.2% of full scale), it can be deemed to be inactive and can be marked as such in the database. Normalized gape, EWMA, EWMV and the GC event flag can be calculated each minute for each bivalve, including the inactive ones. However, the inactive bivalves can be excluded from consideration when determining whether a sufficient number of bivalves are in GC event to signal a toxic exposure. This distinction can be important because temporarily quiescent bivalves are not likely to respond to a toxin in the way that active bivalves would, and their inclusion could affect the accuracy of system-level alarm calculations.

The system can optionally detect filtration activity when gape is recorded at high frequency, identifying rapid but low amplitude spike changes in gape occurring at regular intervals. Since reduction in or cessation of filtration occurs before gape reduction and closing, this behavior can be added to the toxicity signaling algorithm to increase sensitivity and reduce the false signaling rate. The detection of feeding behavior allows improvement in the sensitivity and reduction of false positive rates.

The system can optionally adjust the threshold for the fraction signaling for expected changes in behavior due to changes in water quality parameters at the system level. These adjustments can account for environmental factors that may influence the overall responsiveness of the bivalve population without indicating actual toxicity. The system can monitor auxiliary water quality parameters and can modify the GC event ratio thresholds accordingly to maintain optimal sensitivity while reducing false alarm rates. For example, changes in temperature, dissolved oxygen levels, or other environmental conditions that naturally affect bivalve behavior can trigger corresponding adjustments to the system-level detection thresholds. This adaptive capability can ensure that the system maintains consistent performance across varying environmental conditions while preserving its ability to detect genuine toxic events.

The processor can be configured to normalize the gape measurements to a predefined interval, ensuring consistency and comparability of data across different-sized bivalves. The processor can also be configured to adjust a minimum and maximum gape estimate periodically to account for growth of each organism, allowing for the maintenance of accurate measurement scales over time. The processor can calculate EWMA and EWMV of the adjusted, normalized gape, providing short-term measures of the average gape and the activity level of each bivalve organism. The processor can be further configured to adjust the thresholds in the EWMA/EWMV state-space based on diurnal variations and expected changes in water quality parameters, allowing for a responsive and dynamic monitoring system. The processor can also be configured to apply a machine learning algorithm to refine the detection of deviations indicative of exposure to potentially toxic substances over time, leveraging historical data to improve future detection accuracy.

The detection module can use the EWMA and EWMV as state-space variables to monitor behavior of the bivalve organism and identify deviations that can be indicative of exposure to potentially toxic substances. The alarm system can be configured to generate a system-level alarm when a predetermined fraction of individual bivalves exhibits a behavior consistent with an exposure to a potentially toxic substance, based on thresholds in an EWMA/EWMV state-space. This approach can ensure that the system distinguishes between normal environmental variations and actual contamination events by requiring multiple organisms to simultaneously exhibit the characteristic toxic response pattern.

In certain embodiments, the system can include an automated sampling mechanism. This mechanism can be activated by the alarm system when the system-level alarm is generated, facilitating the collection of water samples for further analysis. The automated response can be a proactive measure in the assessment and management of aquatic toxicity. In particular, the alarm system can include a tiered notification protocol. The protocol can escalate the urgency of an alert based on the number of bivalve organisms exhibiting abnormal behavior and the velocity of change from the normal behavioral state to the abnormal state. The tiered approach can ensure that the severity of the detected toxicity can be accurately communicated, and appropriate responses are initiated. In certain embodiments, the alarm system can comprise a geographic information system (GIS) to visually map a location and spread of detected toxicity events. The system can also convey toxicity signals to responsible personnel, such as on-duty water treatment plant laboratory personnel, by audiovisual alarm, text message, and/or email. The monitoring system can require almost no maintenance, as individual bivalves can survive and provide useful data for years when placed in surface waters with adequate food sources.

The system can be implemented via software. The software can be implemented using Free and Open Source Software (FOSS) architecture. The software implementation can include a database backend implemented in PostGreSQL for data storage and management. The computational code can be written in the R programming language and packaged in an R library to handle the statistical calculations and data processing required for the EWMA and EWMV analysis. Alternatively, the basic calculations can be done efficiently using SQL trigger procedures. The system can include a graphical user interface (GUI) implemented in R/Shiny for display of raw and processed gape data, state space plots for individual bivalves, signaling states of individual bivalves, water quality parameter plots, activity status of bivalves, and signaling status of the overall system. Additionally, the system can incorporate a secure web-based system management interface to enable remote monitoring and control capabilities. The system can also incorporate interactive access to the data using R to analyze the data retrospectively.

The software can handle continuous data streams from multiple sensors simultaneously, processing gape measurements from up to 16 bivalves in real-time while also incorporating auxiliary water quality parameter data. The system can perform complex statistical analysis including autocorrelation functions and state-space trajectory analysis to identify characteristic GC event patterns. The software can also manage the reference sensor data integration to correct for electromagnetic noise and voltage fluctuations in the measurement system.

The processor can be configured to apply a machine learning algorithm to refine the detection of deviations indicative of exposure to potentially toxic substances over time. The machine learning algorithm can leverage historical data to improve future detection accuracy by learning from patterns in the collected gape measurement data. The system can use machine learning approaches to enhance the classification of bivalve behavior patterns and improve the distinction between normal environmental variations and actual contamination events. The machine learning component can be designed to continuously adapt and improve the threshold settings and detection algorithms based on accumulated operational data, thereby reducing false alarm rates while maintaining high sensitivity to actual toxic events.

In operation, the water toxicity detection system can begin with the selection and placement of indigenous bivalve organisms within a sensing enclosure that can allow uniform upflow of water being monitored across the bivalves. Each individual bivalve can be equipped with a dedicated Hall effect sensor using an adhesive system that can hold the sensors firmly in place but allow them to be placed and removed without harming the bivalve. The sensing apparatus can include a movable member that can engage the shell of the mollusk and move when the shell opens and closes, with the magnet positioned on the bottom shell where it is less intrusive and the Hall effect sensor positioned on the top shell to detect the position based on the movement of this member.

The system can continuously monitor auxiliary water quality parameters including temperature, dissolved oxygen, pH, specific conductance, turbidity, and UV/VIS absorbance bands for chlorophyll detection to provide environmental context that help to distinguish between normal behavioral variations due to environmental changes and abnormal behavior indicative of toxic exposure. When the system detects a toxicity signal, the system can convey alerts to responsible personnel, such as on-duty water treatment plant laboratory personnel, by audiovisual alarm, text message, and/or email. The toxicity signal can optionally trigger automated sampling mechanisms for collecting water samples for further analysis. The system can operate with minimal maintenance requirements, as individual bivalves can survive and provide useful data for years when placed in surface waters with adequate food sources, though they can be sacrificed periodically to monitor bioaccumulation of pollutants and replaced with acclimated bivalves.

### EXAMPLES

Example embodiments of the present technology are provided with reference to the several figures enclosed herewith.

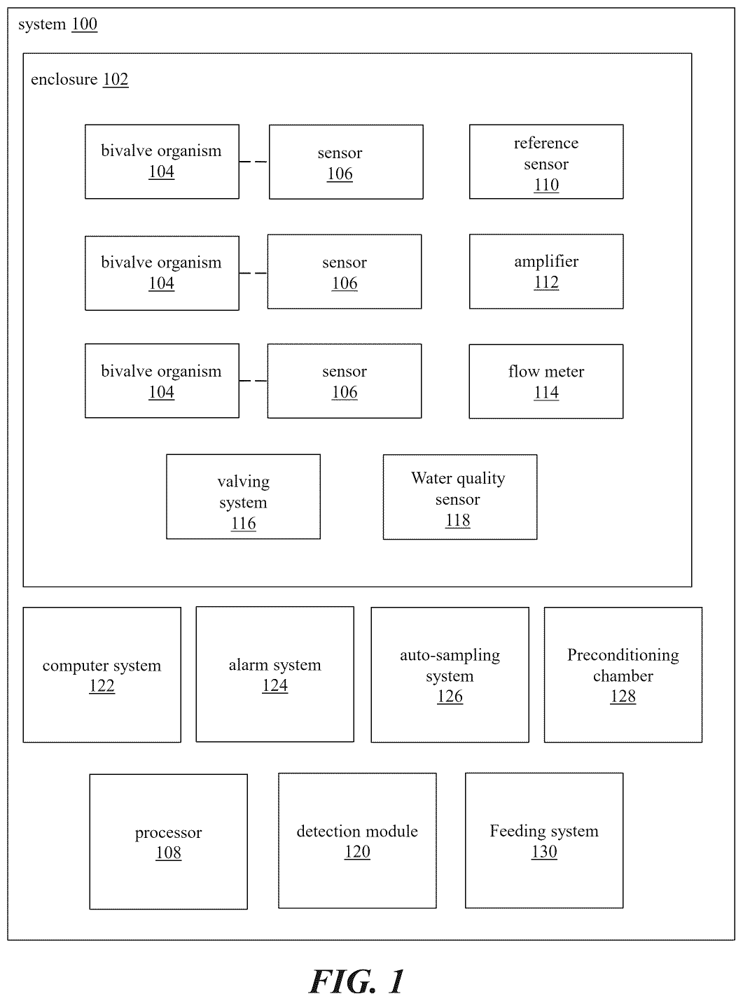

With reference to FIG. 1, a first embodiment of a water toxicity detection system 100 is shown, where the system 100 can include a sensing enclosure 102 containing a plurality of bivalve organisms 104 for monitoring water quality. Each bivalve organism 104 can be equipped with a dedicated Hall effect sensor 106 configured to measure gape behavior and generate corresponding gape measurements. The system 100 can further include a processing module 108 configured to normalize the gape measurements to a predefined interval and calculate an exponentially weighted moving average (EWMA) and an exponentially weighted moving variance (EWMV) of the adjusted, normalized gape.

The sensing enclosure 102 can allow uniform upflow of water being monitored across the bivalves 104, with a valving system 116 and a flow meter 114 to adjust and control the flow of water to the desired velocity. A reference sensor 110 can be positioned within the same enclosure 102 and connected to the same amplifier system 112 as the bivalve sensors 106 to reduce extraneous electromagnetic noise and correct for stray voltages in the tank as well as voltage fluctuations in the amplifier.

The system 100 can incorporate water quality sensors 118 configured to measure auxiliary parameters including temperature, dissolved oxygen, pH, specific conductance, turbidity, and UV/VIS absorbance bands for chlorophyll detection. These auxiliary measurements can be provided to the processing module 108 to enhance the accuracy of toxicity detection and provide environmental context that helps distinguish between normal behavioral variations due to environmental changes and abnormal behavior indicative of toxic exposure.

A detection module 120 can collect data from the sensors 106 and water quality sensors 118, interfacing with a computer system 122 for data storage and system management. An alarm system 124 can be connected to the processing module 108 to receive toxicity signals and generate notifications via audiovisual alarms, text messages, and email alerts to responsible personnel. An automated sampling mechanism 126 can be provided as an optional component that can be triggered by the alarm system 124 for water sample collection when a system-level alarm is generated. The system can also include a pre-conditioning chamber 128 and a feeding system 130.

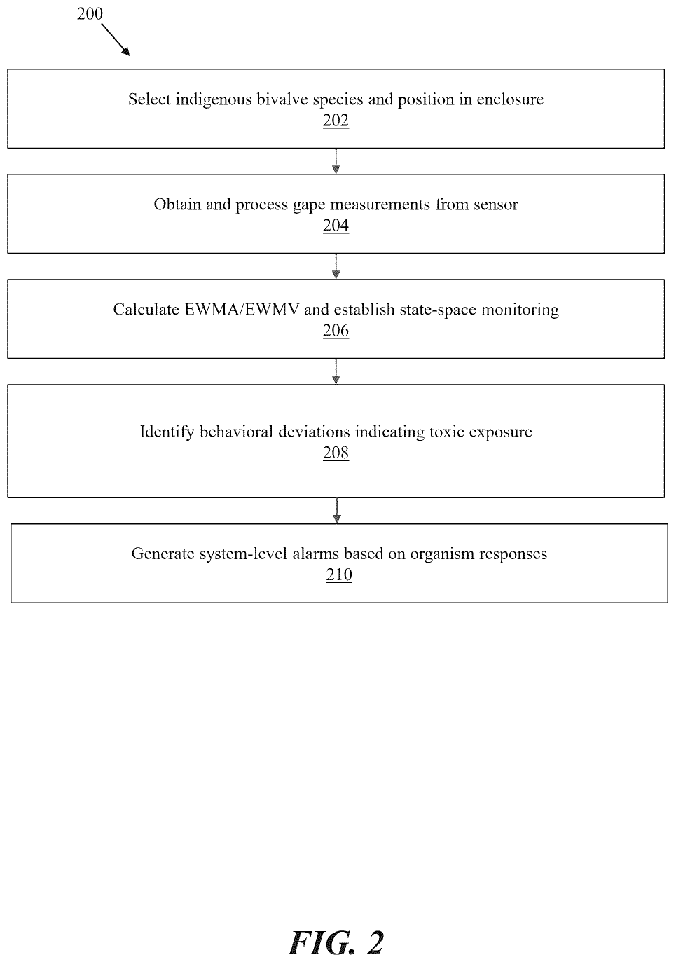

With reference to FIG. 2, a method 200 of detecting toxicity in an aquatic environment can begin at step 202 with selecting and positioning a plurality of bivalve organisms within an enclosure configured to contain water from the aquatic environment, wherein the bivalve organisms can be selected based on indigenous species that are native to the local aquatic environment to be monitored, thereby avoiding introduction of non-native species. This selection process can ensure that the bivalve organisms are already adapted to the local environmental conditions, making the organisms more sensitive and responsive to changes in water quality that could indicate toxic contamination.

At step 204, the method 200 can continue with obtaining and processing gape measurements from each individual bivalve organism using a plurality of sensors positioned within the enclosure at predetermined intervals. This processing can include adjusting the gape measurements using a reference sensor signal to reduce extraneous electromagnetic noise, normalizing the gape measurements from each individual bivalve organism to a predefined interval representing a scale between a completely closed and completely open state, and periodically adjusting minimum and maximum gape values for normalization to account for growth of the individual bivalve organisms.

At step 206, the method 200 can proceed with calculating statistical measures and establishing state-space monitoring by calculating, for each individual bivalve organism, the EWMA of the adjusted, normalized gape as a short-term measure of average gape, and the EWMV of the adjusted, normalized gape as a short-term measure of activity, and determining a state-space based on the EWMA and EWMV for monitoring behavior of each individual bivalve organism.

At step 208, the method 200 can include identifying behavioral deviations indicative of toxic exposure by detecting crossing of certain thresholds in the state-space that can be indicative of exposure to potentially toxic substances. This identification can include detecting gape closing (GC) events that can be characterized by an increase in activity followed by closing and remaining closed, and optionally detecting filtration activity as rapid but low amplitude spike changes in gape occurring at regular intervals when gape is recorded at high frequency.

At step 210, the method 200 can conclude with generating system-level alarms based on active organism responses by determining which individual bivalve organisms exhibit normal patterns of behavior versus inactivity to distinguish between active and inactive organisms, and generating a system-level alarm when a predetermined fraction of the individual bivalve organisms simultaneously exhibit the identified deviations that can be indicative of exposure to potentially toxic substances. The method can optionally include activating an automated sampling mechanism upon generation of the system-level alarm.

### EXPERIMENTAL

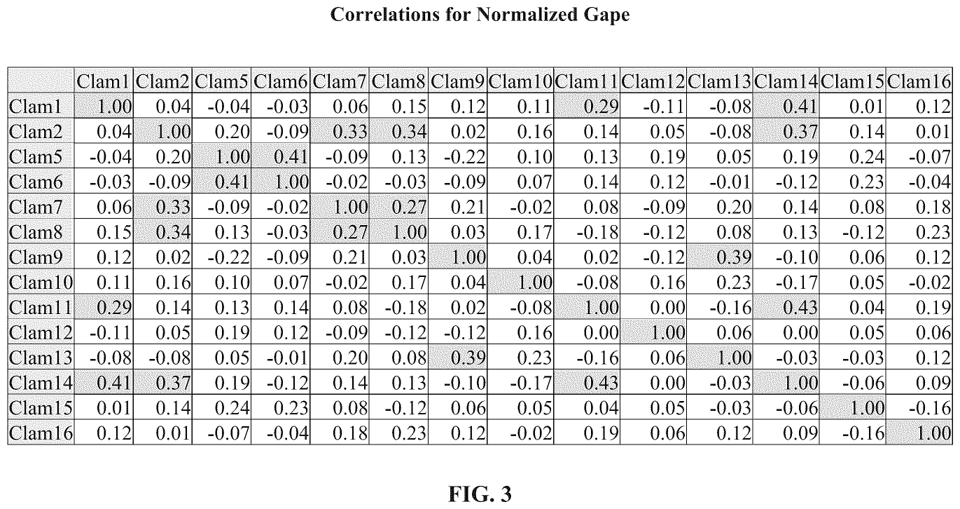

Referring to FIGS. 3-16, models of the methodology and formulations of the present method 200 and system 100 were created and results shown herein verify the monitoring algorithm, provided by the methodology.

In particular, the monitoring algorithm may include: using a reference sensor in the same enclosure and connected to the same signal amplifier and data acquisition hardware as the bivalves; adjusting the raw gape (how open the shell is) signal from the bivalves using a reference sensor signal in order to reduce extraneous electromagnetic noise; this noise could include stray voltages in the enclosure as well as electronic noise in the amplifier and/or data acquisition system; adjusting and normalizing the adjusted gape measurements from individual bivalve organisms at each measurement to the interval [0,1], meaning completely closed to completely open; periodically adjusting the min/max gape estimates to account for the growth of cach organism so that the normalized gape is constrained to the interval [0,1]; using Exponentially Weighted Moving Average (EWMA) of adjusted, normalized gape as a short-term measure of average gape for individual organisms and using Exponentially Weighted Moving Variance (EWMV) of adjusted, normalized gape as a short-term measure of activity for individual organisms; using the EWMA/EWMV as a state-space to describe and monitor the behavior of individual organisms, with the behavior describing a continuous path in this state-space; determining which organisms exhibit normal patterns of behavior versus inactivity at maximum gape in normal environmental conditions, which may indicate illness or death; using thresholds in the EWMA/EWMV state-space to define an event, characterized by an increase in activity followed shortly by closing and remaining closed, that is identified with an individual organism's response to exposure to a potentially toxic substance; optionally adjusting the thresholds in the EWMA/EWMV state-space for individual differences between bivalves, for expected changes in behavior due to changes in water quality parameters, or for the time of day (diurnal variation); using exceedance of a threshold for the fraction of active organisms simultaneously in the above-described event as a signal from the system (versus from individual organisms) of possible toxicity; optionally adjusting the threshold for the fraction signaling for expected changes in behavior due to changes in water quality parameters; optionally, when gape is recorded at high frequency, filtration activity can be detected as a rapid but low amplitude spike change in gape occurring at regular intervals; since reduction in or cessation of filtration occurs before gape reduction and closing, this behavior can be added to the toxicity signaling algorithm to increase sensitivity and reduce the false signaling rate; and sending a message to facility personnel and possibly triggering an autosampler when the system detects a toxicity signal.

Hardware elements of the system can include the following aspects. The algorithm is designed to use data collected from and stored in a hardware system. This hardware system consists of: a collection of bivalves with attached waterproof sensors to detect changes in gape; an example of such a sensor would be a Hall effect proximity sensor; an adhesive system that holds the sensors firmly in place but allows them to be placed and removed without harming the organisms; a reference sensor for noise reduction; an amplifier module to amplify the voltage signals from the gape sensors; a sensing enclosure for the bivalves that allows uniform upflow of water being monitored across the bivalves with valving and a flow meter to adjust and control the flow of water to the desired velocity; one or more water quality sensors used to adjust the expected behavior of the bivalves under routine, nontoxic conditions and to provide additional information in case of a toxicity signal; examples would be conventional water quality parameters, such as pH, temperature, turbidity, specific conductance, dissolved oxygen; a data acquisition device to collect data from the gape sensors and the water quality sensors; and a computer system consisting of one or more computers networked together and providing data storage, data analysis, display of data and toxicity signals, and alerting services.

Optional elements of the hardware system include: a preconditioning chamber for adjusting water temperature to an optimal range and for adding supplemental food if needed for monitoring water with an insufficient amount of phytoplankton; an additional chemistry sensor, for instance a colorimeter or UV/VIS spectrometer; and an additional holding tank or chamber for conditioning bivalves held in reserve as replacements for those that die, are retired, or are sacrificed to monitor bioaccumulation of pollutants, where these bivalves are exposed to the same stream of water as the bivalves in the sensing enclosure.

### Characteristics of Data Used to Develop the Algorithm

Two data sets were used in developing the algorithm. The first is the operational data from the existing online toxicity monitor (OTMs) at the Minneapolis Water Works (MWW) laboratory. The second is the data from the experimental STREAMS facility (ESF) where OTMs were set up in six experimental streams and different doses of low-level copper treatment were applied.

The available data from the MWW real-time OTM consists of gape measurements from 16 bivalves and water quality parameters. The gape measurements vary between 0 and a large positive integer (often >2500). The ESF data consists of only gape measurements from 16bivalves in each of the artificial streams (ESF-1 through ESF-6) for the control and treatment periods and of the Cu dosing.

Because the bivalves vary in size, their gapes must be normalized to the interval [0,1] for analysis. Since the bivalves tend to grow over time, minimum and maximum gape for normalization must be regularly adjusted for each organism as both min and max gapes that are attainable increase over time. For the running min and max, a limited time window of seven (7) days is used, updated at midnight each night. This is believed to be a reasonable period, too short for much growth, but long enough for significant movement over the available raw gape range. The min and max gape are also adjusted as necessary each time data is read, if the data is outside of the min-max gape range.

The normalized gape shows highly significant correlations between bivalves as shown in FIG. 3. There are no correlations less than −0.25. The correlations 0.25 and above are highlighted. The largest is 0.43. There does appear to be significant correlation between organisms. This seems most likely due to similar responses to changing common environmental conditions.

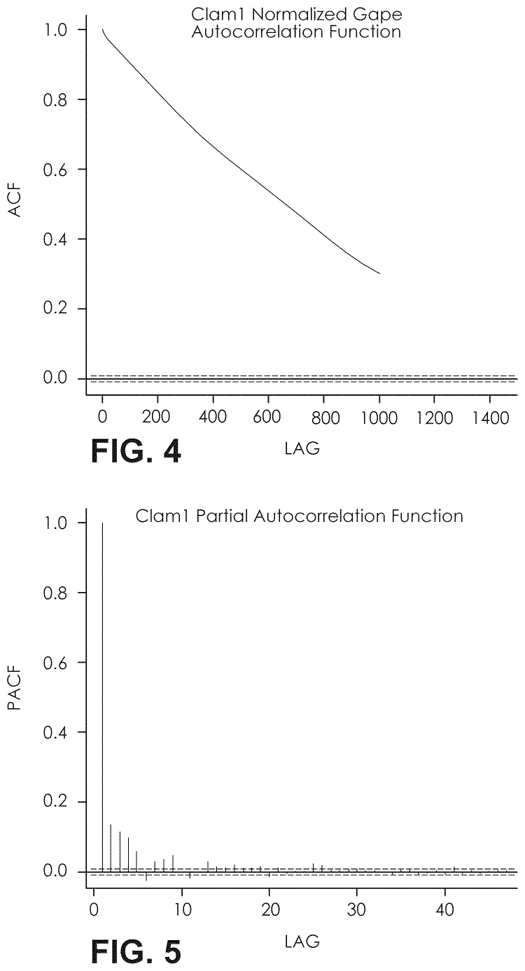

Because the bivalves usually move slowly relative to the data recording rate, the data for each clam is highly autocorrelated. This is true both for raw and normalized gape. The autocorrelation function (ACF) for Clam 1 normalized gape is shown in FIG. 4 to illustrate. This shows a very slowly decaying serial correlation. The autocorrelation is still significant (˜0.1) after one day. The dashed lines show the Box-Ljung limits for statistical significance of the estimated coefficients, which are a function of the length of the time series.

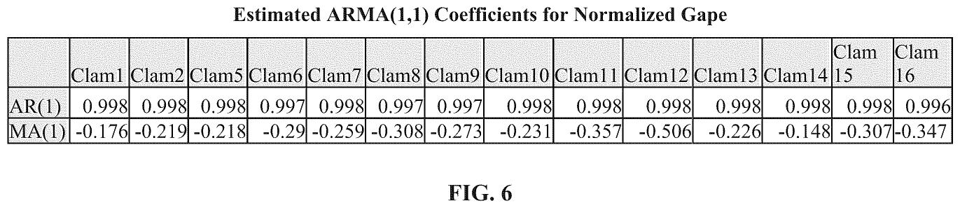

FIG. 5 shows the corresponding partial autocorrelation function (PACF) plot. It is dominated by a lag 1 PACF coefficient that is very close to 1. The PACF coefficients for all the clams are statistically significant out to between the fifth and the tenth (or so) coefficient. This indicates that the process is primarily autoregressive of order 1 (denoted as AR(1)) with the AR(1) coefficient being just barely less than one. It also has a smaller moving average component, making it an autoregressive moving average process of order (1,1), denoted as ARMA(1,1). The estimated ARMA coefficients are given in FIG. 6. As a result of the AR(1) coefficient being very close to one, the normalized gapes form a nonstationary process; they wander all over the interval between 0 and 1 rather than fluctuating around a fixed mean.

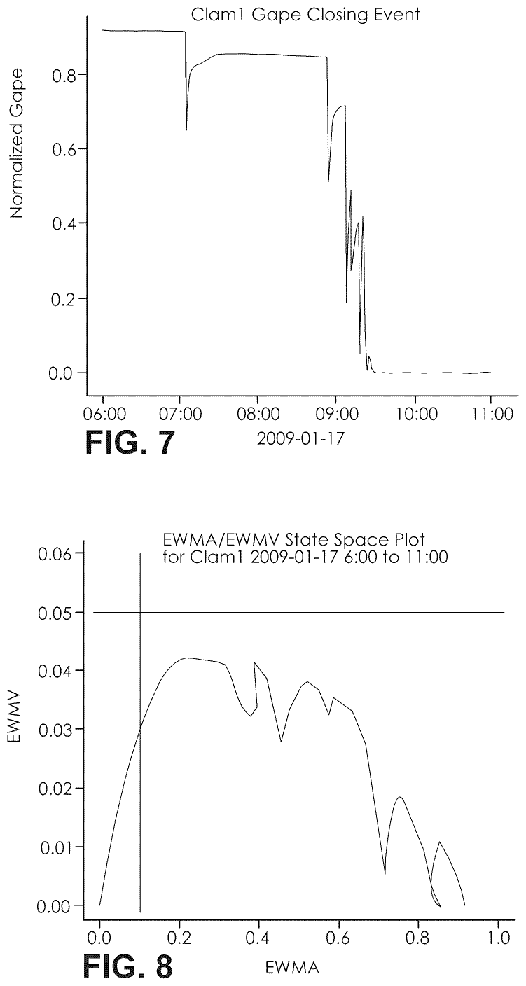

The particular feature of clam gape behavior that indicates irritation or toxicity is an increase in activity, increasing and decreasing the gape (possibly an attempt flush the gills), followed by closing the shell either completely or partially. Typically, the rate of gape change increases dramatically over a period of time from a few minutes to a few hours, then falls to virtually 0 as the clam closes. This behavior is illustrated in FIG. 7. We denote this behavior as a GC event. The algorithm used to identify GC events in the data is described in the next section.

Since this is a natural activity that clams periodically engage in, the signal for irritation or toxicity must be found in a significant change in the behavior of the ensemble of clams. As discussed above, the behavior of individual clams is far from being either identical or identically distributed in a probabilistic sense. As shown by the significant correlations for normalized gape in FIG. 3, the clams do not act independently of each other either. Whether this is due to chemical signaling between clams or to a similar response to changes in environmental conditions is a matter for future research.

### Algorithm for GC Event Detection

The key to the present approach to GC event detection is noting that there is a short-term sharp increase in the local (in time) variance of normalized gape, then a sudden decrease to near zero, accompanied by the local mean gape dropping to a low level. To assess the behavior of local mean and variance of normalized gape, estimators of local mean and variance are needed. For this purpose, Neptune uses the Exponentially Weighted Moving Average (EWMA) and Exponentially Weighted Moving Variance (EWMV) (Carson and Yeh 2008).

The EWMA is defined by the equation yᵢ=wxᵢ+(1−w)yᵢ₋₁

Where xₜ is the normalized gape at time t, w is the weighting parameter, and yₜ₋₁ is the EWMA value at time t−1.

The EWMA is a weighted average (unbiased) of all past observations. The weights sum to one and are exponentially decreasing. As a result, although the EWMA uses all available history, it is quickly responsive to process change, more so as w is increased. This is very important for monitoring normalized gape, since it represents a non-stationary process wandering over the interval of permissible values. The strong positive auto-correlation indicates that an EWMA model will be useful. If normalized gape were a pure autoregressive of order 1 [AR(1)] process, then given the right weight parameter, EWMA would be an optimal predictor in terms of mean squared prediction error (Box, Hunter and Hunter 1978). Another way to view the EWMA is as a low-pass filter that filters out high frequency variation.

The EWMV is defined by the equation

$${v}_{i}^{2} = {w ( {x}_{i} - {y}_{l} )}^{2} + ( 1 - w ) {v}_{i - 1}^{2}$$

where x, is the normalized gape at time t, yₜ is the EWMA at time t, w is the weighting parameter, and v²ₜ₋₁ is the EWMV value at time t−1.

The EWMV is a weighted process variance analogous to the EWMA. It uses all past data but can also be quickly responsive to changes in the process. The EWMA has the role in this formula of the prediction or conditional expected value at time t. Currently, the weight parameters for both the EWMA and the EWMV are 0.1. This can be adjusted to improve signaling characteristics in a specific application.

The signature of a GC event that we are looking for is a substantial increase in the EWMV with decreasing EWMA, followed by EWMA getting small and EWMV going to near 0. This is illustrated in FIG. 8. This is the same clam and time period used in FIG. 7 to illustrate the GC event. The horizontal and vertical lines represent the thresholds for the GC event—less than 0.05 EWMV and less than 0.1 EWMA. Also, in some cases it may be that the clam closes but not completely. The threshold for EWMV also might seem large, but for some clams and at some times the EWMV tends to be much larger. Whether this is due to “nervous” (that is, larger component of high frequency movement) clams or differences in the sensitivity or noise levels of the sensors is unknown. In the future, these thresholds will be estimated for each clam (or other bivalve).

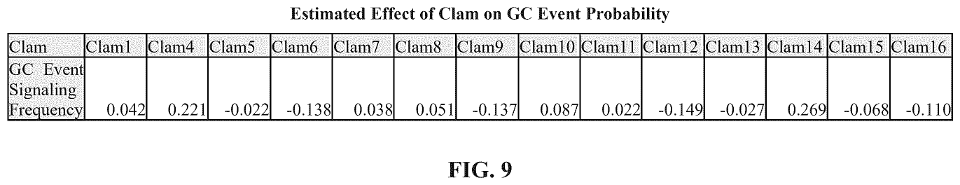

The long-run aggregate frequency of GC events in the MWW data over the period 2008 Dec. 27 to 2009 May 10 is 0.259. However, this rate varies quite substantially among the MWW clams, as shown in FIG. 9, which presents the clam effects on GC event signaling frequency as estimated by median polish, a robust method for analyzing two-way tables (Hoaglin, Mosteller and Tukey 1983). This is a random effect and supports the need for individual EWMA/EWMV signaling thresholds per bivalve.

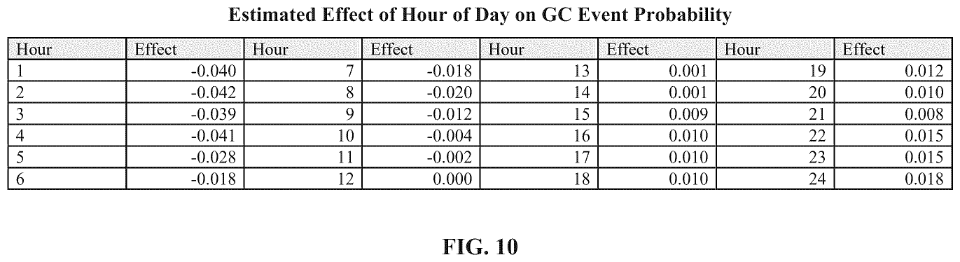

There is also a diurnal effect on the tendency for GC event signaling. The estimated effects on signaling frequency are given in FIG. 10. It is clear that the probability of a GCC event increases from the early morning hours to a maximum in the hour before midnight. The reason for this is unknown. It is also clear that the magnitude of the diurnal effect is less than that of the average difference between clams. In order to improve sensitivity and reduce the false signaling rate, a diurnal adjustment of the EWMA/EWMV thresholds should be examined for statistical and practical significance.

### Assessing Critical Exceedance Thresholds for GC Events

In assessing the ensemble behavior of a group of clams, it is important to distinguish which clams are currently active. Sometimes clams can close and become quiescent or simply be open but fail to change gape for several days, after which they return to normal activity This may have nothing to do with a transient exposure to a toxin or irritant. These temporarily quiescent clams are also not likely to respond to a toxin in the way that active clams would. Therefore, in evaluating the number of clams in the GC event state at any given time, we do not consider the clams that are quiescent. Tendency to periods of quiescence may explain some of the observed differences between clams with respect to GC event frequency.

The number of clams active at any time is random. The active/inactive status of the clams is assessed each day at midnight. If the range of a clam's raw gape measurements over the course of a day does not change by more than 10 units (about 0.2% of full scale), it is deemed to be inactive and is marked as such in the database. Normalized gape, EWMA, EWMV and the GC event flag are calculated each minute for each clam, including the inactive ones. However, the inactive clams are not considered in determining whether a sufficient number of clams are in GC event to signal a toxic exposure.

### No Known Toxic Events—MWW Data

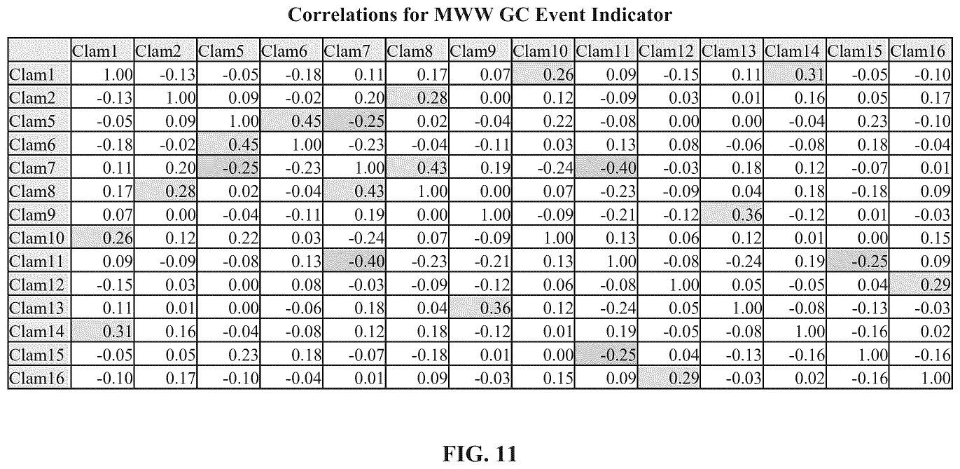

FIG. 11 shows the correlations between the MWW GC event indicator variables. About 20% of them are greater than 0.25 or less than −0.25. Therefore, these indicator variables are not statistically independent.

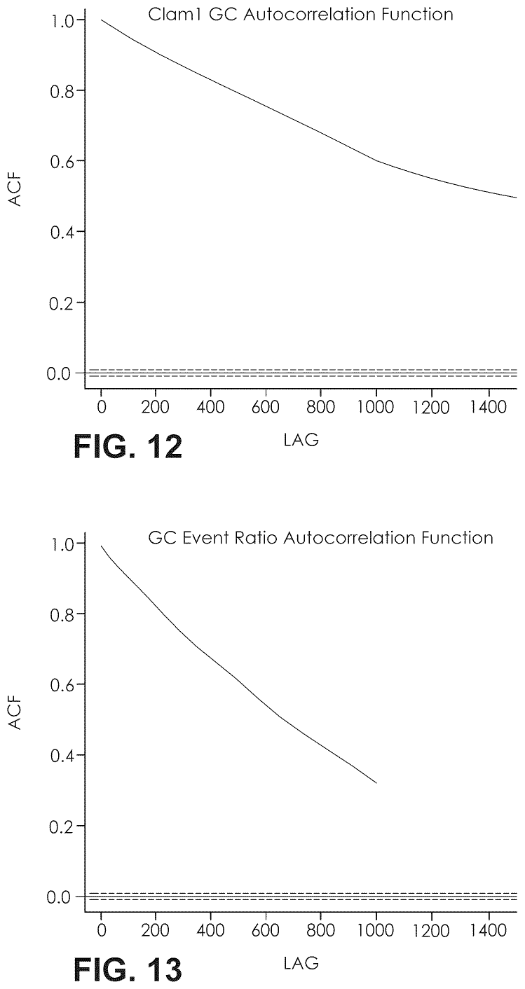

FIG. 12 shows an even more striking departure from the idealized Binomial model. It shows the ACF for the Clam 1 GC event indicator variable. It is even more strongly autocorrelated than the normalized gape with a even more slowly decaying serial correlation. At 24 hours, the lag 1440 autocorrelation is around 0.5 versus 0.1 for the normalized gape.

The relevant statistic to signal a toxic exposure is a function of the number of clams signaling a GC event at a given time or during a window of time and the number available to do so. The first number is a sum of Bernoulli trials, 0-1 random variables, like a Binomial random variable. However, that is all that it has in common with a Binomial random variable. A Binomial random variable is the sum of a fixed number of independent and identically distributed (same probability of success) Bernoulli random variables. The random sum, which is the number of concurrent GC events at a given time, is a sum of a random number (number of active clams) of Bernoulli trials which do not all have the same probability of occurring, which which are highly dependent over time and are dependent between clams.

Therefore, although the number of clams in the GC event state may appear to have similarities to a Binomial random variable, at least conditional on the number of active clams, in fact, it has a very different distribution. Until a suitable distributional model can be found, the statistical characteristics of this quantity must be determined empirically.

The most useful simple statistic to monitor based on these quantities is the ratio of clams in a GC event state to the number of active clams. Let this quantity be denoted by the term ‘GC event ratio’. It is not surprising that the ACF of the GC event ratio has a very large and slowly decaying serial correlation as shown in FIG. 13.

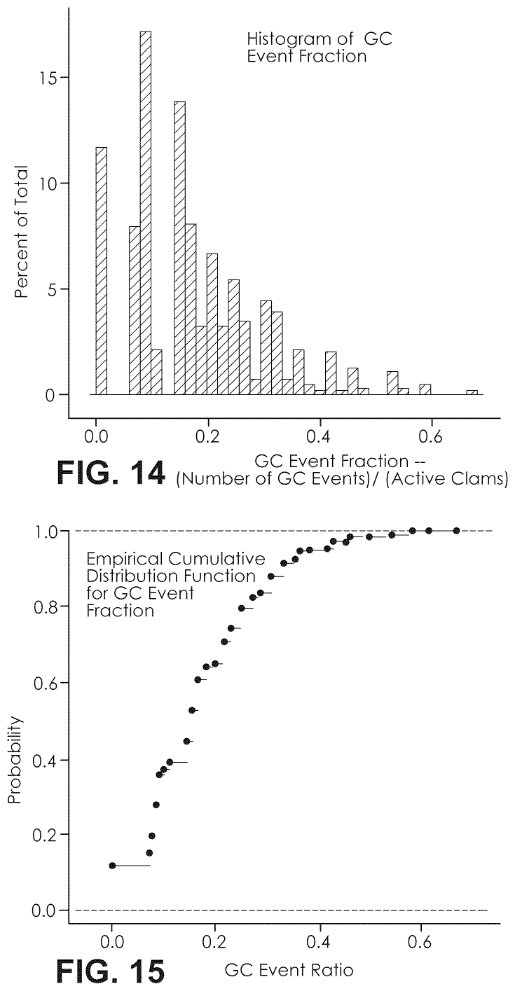

FIG. 14 presents a histogram of the GC event ratio. Since the number of active clams is always 16 or less, only certain values of the ratio are possible. In fact, these values are in the set i/j, i=0, . . . , j; j=1, . . . ,16. FIG. 15 shows a plot of the empirical cumulative (probability) distribution function (ECDF) for the GC event ratio. From this we can see that a GC event ratio of 0.4 is exceeded about 5% of the time and a GC event ratio of 0.6 is exceeded about 0.1% of the time.

Consider setting a simple threshold τ for GC event ratio that functions as an alarm level. If the GC event ratio exceeds τ, we say there has been a ‘toxic event detection’. Then FIGS. 14 and 15 only tell part of the story in that they inform us not about the frequency of alarms but about the fraction of time each month that the system is in the alarm state, as a function of the chosen alarm level. But, when the alarm level has been exceeded, we consider it as one alarm whether that state continues for 1 minute or 5 hours. Therefore, in order to look at the expected number of alarms each month as a function of the alarm threshold, we need to consider the run length distribution of the indicator of the GC event ratio exceeding the threshold.

Looking at the run length distribution of alarm exceedances, we can estimate that setting the alarm threshold to 0.4 for the MWW data would give about 15 alarms a month with the system in an alarm state about 5% of the time, setting the alarm threshold to 0.5 for the MWW data would give about 10 alarms a month with the system in an alarm state about 1.5% of the time, setting the alarm threshold to 0.6 for the MWW data would give about 6 alarms a month with the system in an alarm state about 0.2% of the time.

There are no GC event ratio values greater than about ⅔. Setting the alarm threshold to just under ⅔ for the MWW data would give about the same results as setting it at 0.6. So, if a GC event ratio alarm level above ⅔ is able to detect low level toxic exposures in the ESF data, it will be very useful.

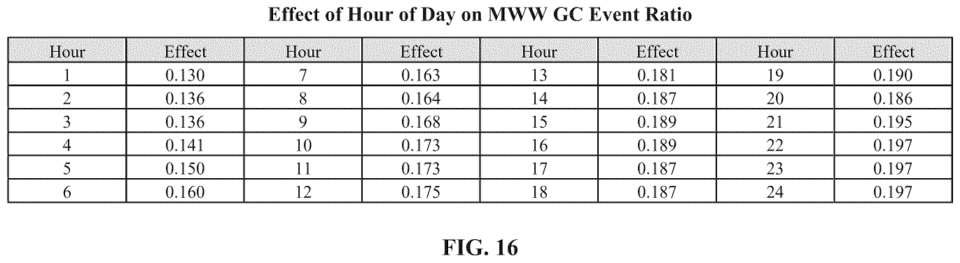

The diurnal effect on the GC event signaling frequency of individual clams carries over into a diurnal effect on the GC event ratio, the ratio of the number of active clams in the GC event state to the number of active clams at each point in time. This is illustrated in FIG. 16. These estimates were produced using ANOVA. The model was highly significant (p-value ˜0) and diagnostic plots looked good. Based on this, the alarm threshold could be varied based on the time of day.
Box, G. E. P., W. G. Hunter, and J. S. Hunter (1978) Statistics for Experimenters—An Introduction to Design, Data Analysis, and Model Building. New York, NY, John Wiley & Sons.
Carson, P.K. and A.B. Yeh. (2008) Exponentially Weighted Moving Average (EWMA) Control Charts for Monitoring an Analytical Process. Ind. Eng. Chem. Res. 2008, 47, 405-411.
Dobson, A. J. (1983) Introduction to Statistical Modeling. Chapman and Hall, New York.
Draper, N. and H. Smith (1981) Applied Regression Analysis: 2nd Edition. John Wiley and Sons, New York.
Hoaglin, D. C., Mosteller, F. and J. W. Tukey (1983) Understanding Robust and Exploratory Data Analysis. John Wiley, New York.
McCullagh, P. and J.A. Nelder (1989) Generalized Linear Models: 2nd Edition. Chapman & Hall, London.
Pankratz, A. (1985) Forecasting with univariate Box-Jenkins models: concepts and cases. John Wiley. New York. 562 pages
R Development Core Team (2009) R: A language and environment for statistical computing. R Foundation for Statistical Computing, Vienna, Austria. ISBN 3-900051-07-0, URL http://www.R-project.org.
Venables, W. N. and Ripley, B. D. (2002) Modern Applied Statistics with S. Fourth Edition. Springer-Verlag.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A system for detecting toxicity in an aquatic environment, comprising:
a plurality of bivalve organisms positioned within the aquatic environment;
a plurality of sensors, each sensor configured to measure a gape of an individual bivalve organism and generate a corresponding gape measurement for that individual bivalve organism;
a processor configured to:
normalize the gape measurement from each individual bivalve organism to a predefined interval to generate a normalized gape;
calculate, for each individual bivalve organism, an exponentially weighted moving average (EWMA) and an exponentially weighted moving variance (EWMV) of the normalized gape;
a detection module configured to:
use the EWMA and EWMV as state-space variables to monitor behavior of each individual bivalve organism;
identify a predetermined deviation in behavior of each individual bivalve organism that is indicative of exposure to an environmental stressor;
an alarm system configured to generate a system-level alarm when a predetermined fraction of the individual bivalve organisms exhibits the predetermined deviation.

2. The system of claim 1, wherein the plurality of sensors comprises Hall effect sensors configured to detect movement of a shell of each bivalve organism.

3. The system of claim 1, further comprising an amplifier system in communication with the processor and configured to amplify a voltage signal from each sensor.

4. The system of claim 1, further comprising an enclosure housing the plurality of bivalve organisms.

5. The system of claim 4, further comprising a reference sensor positioned within the enclosure with the plurality of bivalve organisms, wherein the reference sensor is used to adjust a raw gape signal from each individual bivalve organism to minimize electromagnetic noise.

6. The system of claim 4, wherein the enclosure includes valving and a flow meter to adjust and control a flow of water through the enclosure to a predetermined velocity.

7. The system of claim 4, further comprising an auxiliary sensor positioned within the enclosure and configured to measure a water quality parameter.

8. The system of claim 7, wherein the water quality parameter is a member selected from a group consisting of: a temperature, an amount of dissolved oxygen, a pH, a specific conductance measurement, a turbidity measurement, a UV/VIS absorbance band for chlorophyll detection, and combinations thereof.

9. The system of claim 1, wherein the detection module identifies abnormal behavior through detection of a gape closing (GC) event characterized by an initial increase in EWMV accompanied by a decrease in EWMA, followed by a decrease in EWMA and the EWMV approaching zero.

10. The system of claim 1, wherein the processor is further configured to periodically adjust minimum and maximum gape estimates to account for growth of each bivalve organism.

11. The system of claim 1, further comprising an automated sampling mechanism activated by the alarm system upon generation of the system-level alarm.

12. The system of claim 1, wherein the processor is configured to apply a machine learning algorithm to refine the detection of deviations indicative of exposure to the environmental stressor over time.

13. The system of claim 1, wherein the alarm system includes a tiered notification protocol that escalates urgency of an alert based on a number of bivalve organisms exhibiting the predetermined deviation and the rate at which this deviation occurs.

14. A method of detecting toxicity in an aquatic environment, comprising:
positioning a plurality of bivalve organisms within an enclosure configured to contain water from the aquatic environment;
obtaining a corresponding gape measurement from each individual bivalve organism using a plurality of sensors positioned within the enclosure;
normalizing the gape measurement from each individual bivalve organism to a predefined interval to generate a normalized gape;
calculating, for each individual bivalve organism, an exponentially weighted moving average (EWMA) and an exponentially weighted moving variance (EWMV) of the normalized gape;
monitoring behavior of each individual bivalve organism using the EWMA and EWMV as state-space variables;
identifying a predetermined deviation in behavior of each individual bivalve organism that is indicative of exposure to a potentially toxic substance;
generating a system-level alarm when a predetermined fraction of the individual bivalve organisms exhibits the predetermined deviation.

15. The method of claim 14, further comprising adjusting the gape measurements using a reference sensor signal to minimize electromagnetic noise.

16. The method of claim 14, further comprising periodically adjusting minimum and maximum gape values for normalization to account for growth of each bivalve organism.

17. The method of claim 14, wherein identifying the predetermined deviation comprises detecting a gape closing event characterized by an initial increase in EWMV with a decrease EWMA, followed by decrease in EWMA and the EWMV approaching zero.

18. The method of claim 14, further comprising selecting the plurality of bivalve organisms based on an indigenous species that is native to the local aquatic environment to be monitored, thereby avoiding introduction of a non-native species.

19. The method of claim 14, further comprising monitoring auxiliary a water quality parameter including a member selecting from the group consisting of a temperature, a dissolved oxygen level, a pH, specific conductance value, a turbidity measurement, a UV/VIS absorbance band for chlorophyll detection, and combinations thereof.

20. The method of claim 14, wherein obtaining the gape measurement comprises using a plurality of Hall effect sensors configured to detect movement of a shell of each bivalve organism.
